(19) United States
(12) Patent Application Publication
(10) Pub. No.: US 2009/0118818 A1
(43) Pub. Date: May 7, 2009

(54) ENDOPROSTHESIS WITH COATING

(75) Inventors: Aaron Foss, Playmouth, MN (US); Raed Rizq, Maple Grove, MN (US); Daniel Vancamp, Elk River, MN (US)

Correspondence Address: FISH & RICHARDSON PC, P.O. BOX 1022, MINNEAPOLIS, MN, 55440-1022, US

(73) Assignee: Boston Scientific Scimed, Inc., Maple Grove, MN (US)

(21) Appl. No.: 11/934,296

(22) Filed: Nov. 2, 2007

(51) Int. Cl. A61F 2/82; A61L 27/28

(52) U.S. Cl. 623/1.42; 623/1.46; 427/2.25

(57) ABSTRACT

An endoprosthesis such as a coronary stent includes a surface region that defines a depression in the form of a channel, the depression having an interior surface, and an enhanced roughness on the interior surface.

### TECHNICAL FIELD

This disclosure relates to endoprostheses, such as stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire contents of which are hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

Passageways containing endoprostheses can become re-occluded. Re-occlusion of such passageways is known as restenosis. It has been observed that certain drugs can inhibit the onset of restenosis when the drug is contained in the endoprosthesis. It is sometimes desirable for an endoprosthesis-contained therapeutic agent, or drug to elute into the body in a predetermined manner once the endoprosthesis is implanted.

### SUMMARY

In an aspect, the invention features an endoprosthesis, comprising a channel on a surface region. The channel includes a ceramic coating on at least a portion of its interior surface. The coating has a defined grain morphology.

In another aspect, the invention features a method of forming an endoprosthesis, comprising forming a channel on the endoprosthesis, treating the interior surface of the channel such that at least a portion of the surface has an Sdr of 30 or greater, and applying a polymer, e.g. a polymer containing a drug to the channel.

Embodiments may also include one or more the following features. The channel can include a polymer containing a drug adhered to the ceramic. The polymer can be swellable on exposure to body fluid. The coating can have an Sdr of about 3 or more. The ceramic can include oxides and nitrides of iridium, titanium, zirconium, hafnium, niobium, tantalum, ruthenium, platinum, and aluminum. The ceramic can be IROX. The coating can have a thickness of about 10 to 500 nm. The surface region can be the abluminal surface of a stent wall. The channel can have a depth of about 50% or less of the thickness of the stent wall. The polymer can have a thickness smaller than the depth of the channel.

Embodiments may also include one or more the following features. The channel can be formed by a laser ablation process. The channel can be formed in the body of the endoprosthesis. A coating can be formed on the endoprosthesis and the channel can be formed in the coating. The coating can be a ceramic. The ceramic can be formed by pulsed laser deposition (PLD). The polymer can be applied by dipping, spraying, or vapor deposition. The interior surface can be treated by etching. The interior surface can be treated by depositing a ceramic layer. The ceramic layer can be applied by PLD. The ceramic can have a defined grain morphology. The ceramic can be IROX.

Embodiments may include one or more of the following advantages. Continuous or discrete depressions (e.g., in the form of channels) and/or ridges can provide a cavity to contain biologically active substances, such as drugs as well as provide more surface areas. The drug may be provided in a carrier, e.g. a polymer that is swellable. The cavity into which a polymer that might swell also creates forces that confine the polymer within the cavities. The depression (e.g., in the form of a channel) defined in a surface of a medical device (e.g., a stent) or the channel defined by ridges protects the drugs during delivery of the device into the body. During delivery, e.g., via a catheter, drugs and drug eluting polymers located within such depressions remain generally undisturbed and in place, while substances located on a generally flat surface of currently available medical devices are exposed and thus subject to shear forces that can strip the substances off the surface. Roughening surfaces of the depressions and ridges can further help confining drugs in place by enhancing adhesion of the drug eluting polymers to the surfaces. The surfaces can be roughened by forming a coating with predetermined texture or surface morphology over the select surface regions of the depressions, ridges, and/or stent. The coating can be formed of a ceramic, e.g. IROX, which can have therapeutic advantages such as reducing the likelihood of restenosis and enhancing endothelialization. The coating can be formed by physical vapor deposition process, such as PLD. The surfaces can also be roughened directly by, e.g. chemical etching, such as electrochemical etching lasers, ion bombardment, or macroblasting. Stents can be formed with high loadings of drug in the depressions or channels formed by the ridges (e.g., a drug reservoir) on select portions, such as the abluminal surface. The drug can be loaded in large amount.

Still further aspects, features, embodiments, and advantages follow.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIGS. 3A-3C are cross-sectional views of a stent wall.

FIG. 4 is a cross-sectional schematic of a stent wall.

FIGS. 5A and 5B are cross-sectional schematics of a stent wall.

FIG. 6 is a flow diagram illustrating manufacture of a stent.

FIG. 7 is a schematic of a PLD system.

FIG. 8A and 8B are FESEM images of a stent wall surface.

FIG. 9 is an FESEM image of an etched metal surface.

### DETAILED DESCRIPTION

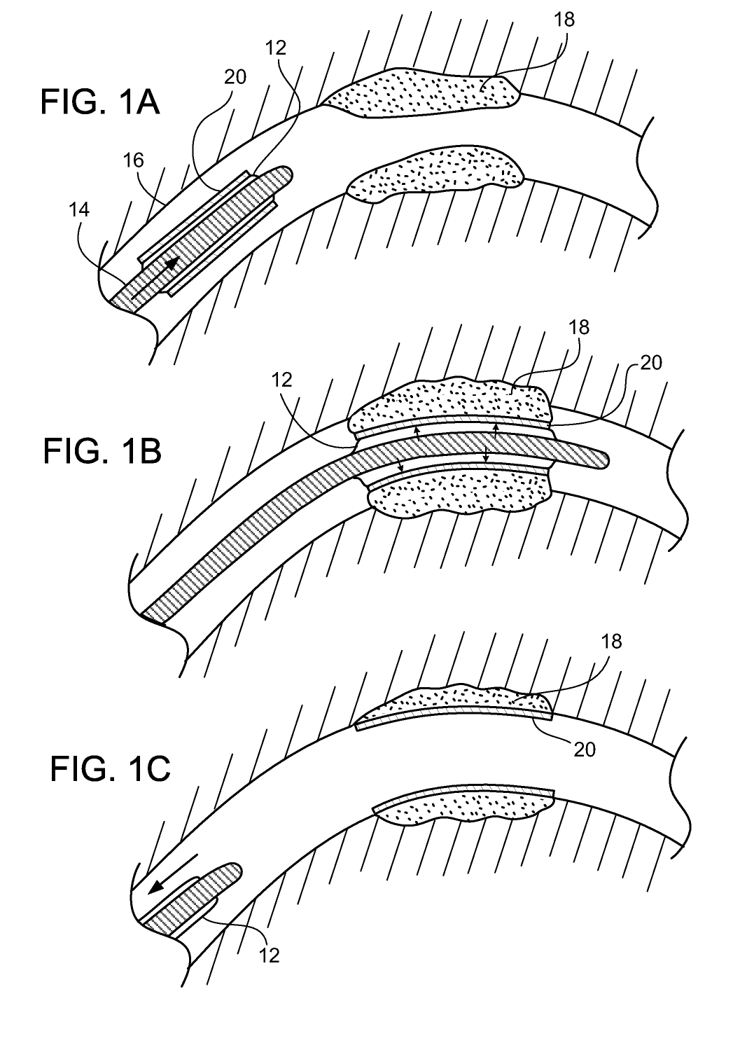

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

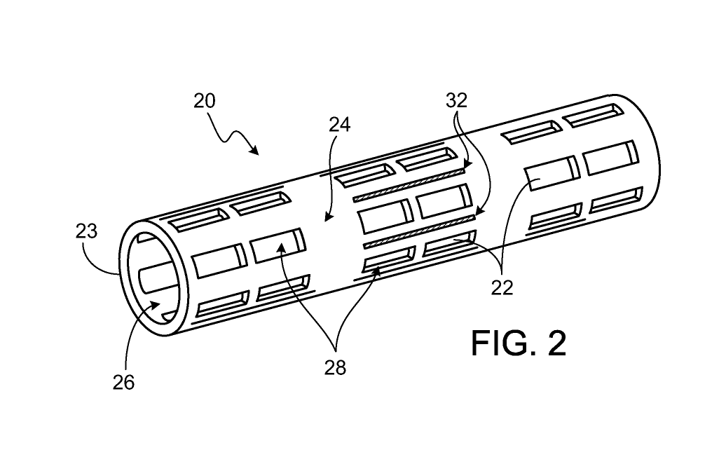

Referring to FIG. 2, the stent 20 includes a plurality of fenestrations 22 defined in a wall 23. Stent 20 includes several surface regions, including an outer, or abluminal, surface 24, an inner, adluminal, surface 26, and a plurality of cutface surfaces 28. At least one of the surface regions, e.g. the abluminal surface further includes a plurality of depressions 32 in the form of channels that extend generally along the longitudinal axis of the stent (e.g., longitudinal orientation with respect to the normal blood flow) and/or the circumferential axis of the stent. The channels 32 are defined within the stent wall 23 but are not completely through the wall thickness. The stent can be balloon expandable, as illustrated above, or a self-expanding stent. Examples of stents are described in Heath '721, supra.

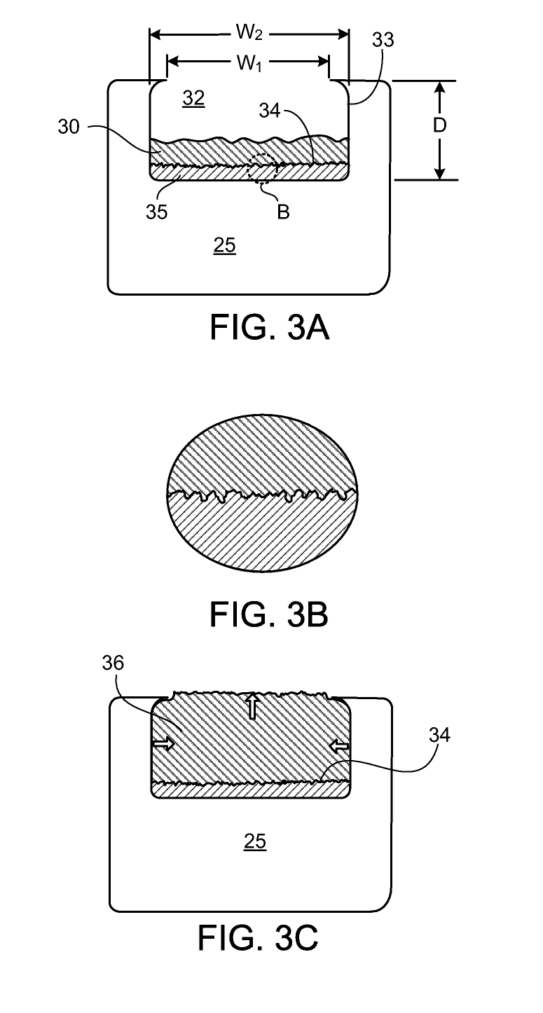

Referring to FIG. 3A, a cross-sectional view, a stent wall 23 includes a stent body 25 formed, e.g. of a metal, and includes a depression, e.g. in the form of a channel generally along the longitudinal axis of the stent. The channel 32 can be defined by an opening with a width W₁ and the interior surfaces: a bottom surface 35 opposite to the opening, and two generally parallel side surfaces 33. The channel 32 can be used to accommodate a biocompatible substance, e.g., a drug-containing polymer 36. A coating 34, formed, e.g. of a ceramic, covers at least one interior surfaces of the channel. Referring to FIG. 3B, a greatly enlarged view of the region B in FIG. 3A, the coating 34 has predetermined texture or surface morphologies that enhances the adhesion of drug-containing polymers to the channel, as will be discussed further below.

In this embodiment, the thickness of the polymer layer 36 is less than the depth of the depressions such that the coating is protected from sheer forces, e.g., during handling and delivery into the body. Because the devices described herein can minimize loss of the biocompatible substances, relatively lower amounts of the substances can be provided in the stent. The drug-containing polymer can have a reduced thickness, for example, the stents described herein can include biocompatible substance having a thickness of about 5 μm or less, e.g. about 3 μm, containing biodegradable polymers, and having up to an 80% or more, e.g. 90% or 100% release ratio of a biologically active substance, as such substance is now protected during delivery.

Referring to FIG. 3C, in embodiments, the polymer layer 36 can swell upon exposure to fluid, e.g. upon exposure to body fluid on implantation in a vessel, which causes the coating to fill the channel 32. The amount of swelling can be such that the polymer is compressed (arrows) by the walls of the channel. The channel therefore controls the expansion of the coating, controls the density of drug in the polymer and/or compresses the coating against adjacent tissue (double arrow) through channel opening.

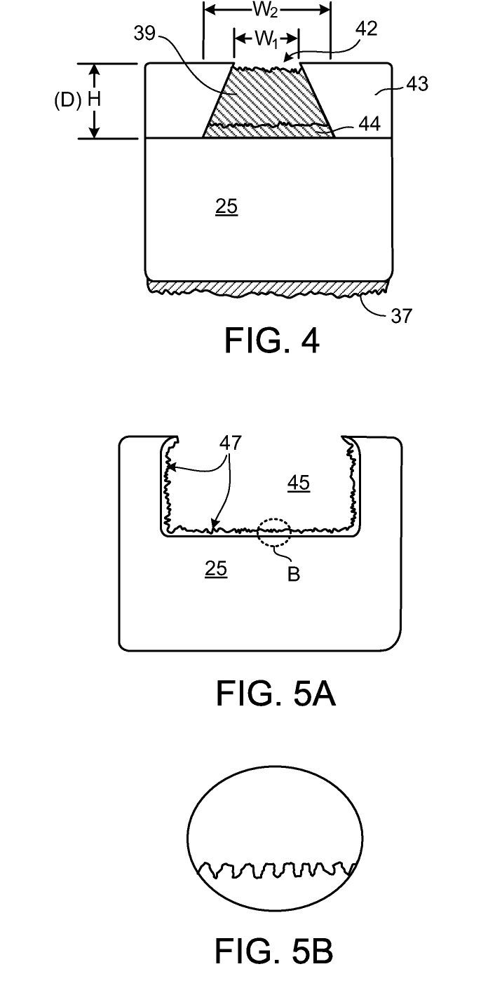

Referring to FIG. 4, in other embodiments, a channel 42 is formed by depositing material onto a stent body, e.g. the abluminal surface 24, and forming at least two generally parallel ridges 43. The ridges can be formed of the same material as that of the stent body 25, or a different material, e.g., a ceramic or polymer. A coating 44, formed, e.g. of a ceramic, covers at least some portions of the abluminal surface 24 within the channel and/or some portions of the ridges. The channel can also be used to accommodate a biocompatible substance, e.g., a drug-containing polymer 39, in this example illustrated as a substantially non-swellable polymer that substantially fills the channel 42. The coating 44 has predetermined textures or surface morphologies that enable, e.g., the adhesion enhancement of drug-containing polymers to the channel. The ridges 43 can have undercutting sides as shown in the figure or have lips or ledges so that the biocompatible substance can further be confined within the channel 42 that are enclosed by the ridges. In the embodiment illustrated, the stent includes a second ceramic coating 37 on its luminal surface, which may be the same or a different composition or morphology than the coating 44. For example, the coating 37 may have a less rough morphology selected for enhancing endothelialization of the stent. A rough coating 44 can be deposited only in the channels (e.g. by making the luminal, abluminal and/or cutface surfaces include less rough coating.

In embodiments, the depth of channel D can constitute on average up to about 50% (e.g., about 35%, or 25%, or 15%, or 10%, or 5% or less) of the thickness of the stent wall 23, in which the channel is defined. In embodiments, the channel width W₂ or the average distance of the two parallel side walls of the channel is about 50% or less than the width of the stent body region (e.g., a strut region) on which the depression is located and/or greater than the opening width W₁. As a result, the channel has lips or ledges that can further confine the biocompatible substance inside the channel through e.g., mechanical retention. The channel can be continuous or discrete along the stent axes. The channel can have a perimeter of various shapes, e.g., a generally rectangular shape as shown in FIG. 3, or an ellipse, or trapezoid, or an irregular shape. In embodiments, the drug containing polymer can be swellable or non-swellable. For example, a swellable polymer can swell upon exposure to fluid to 100% or more (i.e. 100% refers to swelling to double its initial thickness), e.g., about 200%, 300% or 400% of its initial thickness. The polymer containing the drug can be bioerodible or biostable. Suitable polymers are described further below. Coatings 34 and 44 can be formed by physical vapor deposition (“PVD”) processes. The thickness of the coatings is less than the depression depth and/or the ridge height, e.g. about 0.2% to 10% of the depth D and/or height H. The coatings are formed of a ceramic or metal that is selected for compatibility with materials forming depressions and/or ridges. The morphologies and roughness of the coatings can be selected, as will be described further below.

Referring to FIGS. 5A and 5B, in another embodiment, the morphology of the interior of a channel 45 is modified by forming a high roughness surface 47 on the stent body, 25 or a metal coating (not shown) applied on the stent body. The high roughness surface etching can be formed by, e.g., electrochemical etching.

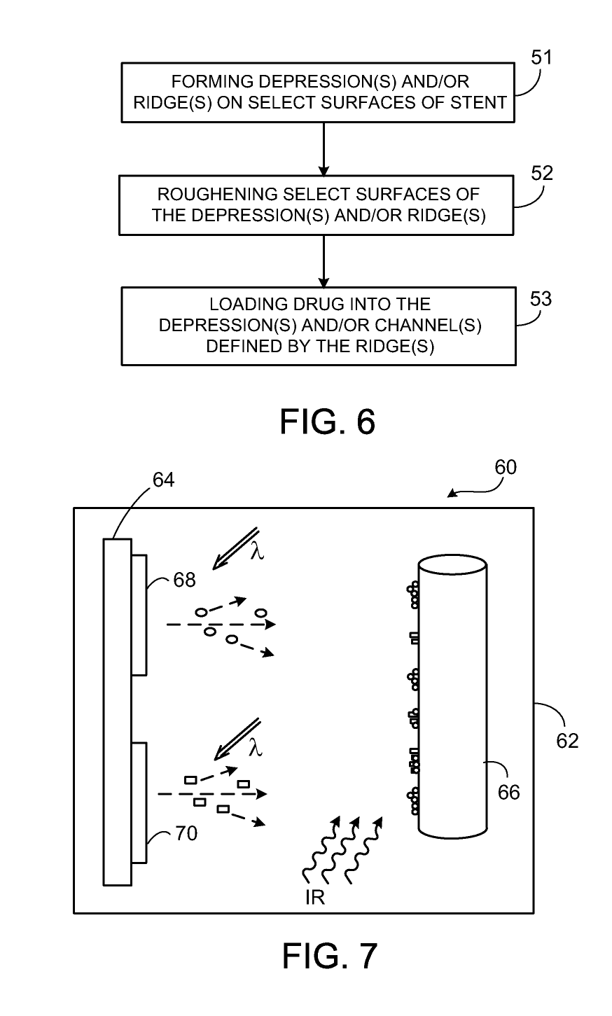

Referring to FIG. 6, the stent is formed by first forming depressions and/or ridges on select surfaces of the stent (step 51). Next, the select surfaces of the depressions and/or ridges are provided with a ceramic or metal coating to the depressions or ridges on the stent, e.g. by pulsed layer deposition (“PLD”) or etching (step 52). Finally, a drug-containing polymer is applied into the depressions or channels formed by the ridges (step 53).

Referring particularly to step 51 in FIG. 6, depression 32 as shown in FIG. 3 can be made by a variety of methods, e.g., by laser ablation process, micromaching, laser-assisted chemical etching, dry etching, or wet etching, e.g., anisotropic etching. For example, the depression 32 can be generated by laser, e.g., ultra-short pulsed laser, e.g., a laser system delivering femtosecond pulses in the ultraviolet range (about 248 nm), e.g., short-pulse dye excimer hybrid laser delivering about 500-fs pulses at 248 nm. Bekesi et al., Appl. Phys. A 76:355-57, 2003. The depression can also be generated by a UV laser, e.g., 248 nm or 193 nm laser, having pulse length in the nanosecond range. The depression can be generated with an ultra-short laser having pulse length of sub pico, femto, or even attosecond length, operating at various wavelengths, e.g., visible, infrared, or near infrared. Description of the depressions in the surfaces of the stent and methods of forming the depressions is further provided, e.g., in Weber et al., U.S. Provisional Application No. 60/844,471, filed Sep. 14, 2006, the entire disclosure of which is hereby incorporated herein.

Referring to FIG. 6, step 52, in embodiments, the ceramic or metal coating is provided over depressions or ridges by physical vapor deposition, such as PLD, more detail of which is described later in this disclosure. In embodiments, masks can be applied to the surfaces outside the depression or channel to shield the regions from the deposit. In other embodiments, it may be desirable to remove the coating from the surfaces outside the depression or channel, e.g., by grinding or laser ablation, leaving the coating mainly inside the depression or channel. In certain embodiments, the surfaces outside the depression or channel can be coated with a material the same or different from that inside the depression or channel, such as a material of different composition, or a material of the same composition but different in surface morphology.

In other embodiments, the surface of the depression is treated by chemical etching the select stent surface. For example, a stent formed of an alloy, e.g., a stainless steel alloy stent, can be electrochemically etched in a solution, e.g., sulfuric acid, to form surfaces with texture of roughness in the range of a few nanometers to a few micrometers. Other methods can also be used to modify surfaces of the depression and ridges to increase roughness, such as laser microblasting, ion bombardment, e.g. with argon or helium, or electroplasma treatment. Description of forming porous surface regions through dealloying is provided in ______ [Attorney docket number 10527-820001].

Referring to FIG. 6, step 53, in embodiments, the drug may be co-applied to the stent with the polymer, e.g., the drug-containing polymer is loaded into the depressions or channels by dip coating or spraying the stent in solution of a drug and polymer or polymer precursor and drying under low temperature, e.g. ambient conditions. The drug is as a result precipitated into the depressions or channels. The loading can be facilitated by repeatedly dipping and drying while the stent substrate is cooled under evacuated conditions. Other techniques such as rolling, pulsed laser deposition (“PLD”), pressing, brushing, or laminating can also be applied to load the drug-containing polymer to the depressions or channels. In embodiments, the drug may be loaded into the polymer in a separate step by, e.g., absorption of the polymer, after the polymer is applied to the depressions or channels.

Referring to FIG. 7, the PLD system 60 includes a chamber 62 in which is provided a target assembly 64 and a stent substrate 66, such as a stent body or a pre-stent structure such as a metal tube. The target assembly includes a first target material 68, such as a ceramic (e.g., IROX), or a precursor to a ceramic (e.g., iridium metal), or a metal, e.g. stainless steel and a second target material 70, such as a drug. In some embodiments, the target assembly includes only one target material. Laser energy (double arrows) is selectively directed onto the target materials to cause the target materials to be ablated or sputtered from the target assembly. The sputtered material is imparted with kinetic energy in the ablation process such that the material is transported within the chamber (single arrows) and deposited on the stent 66. In addition, the temperature of the deposited material can be controlled by heating, e.g. using an infrared source (squiggly arrows). The surface morphologies of the ceramic or metal coating can be controlled by varying the film thickness, the laser power, the total background pressure, and the partial pressure of oxygen, or the oxygen to argon ratio if reactive PLD is utilized. Coating thickness is controlled by controlling deposition time. Higher laser energies can provide larger cluster sizes.

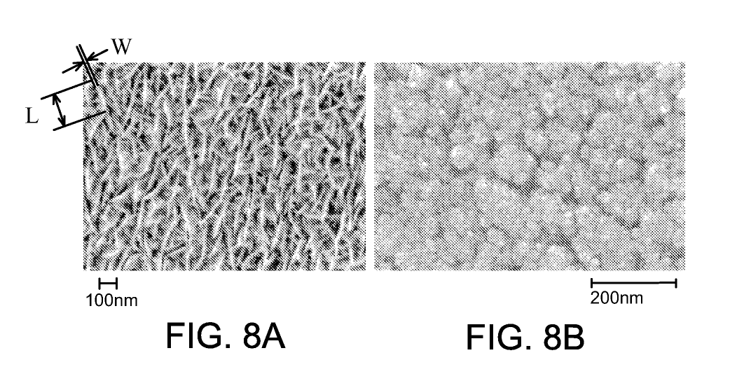

In particular, a ceramic coating has a select morphology or roughness that enhances the adhesion of the drug-eluting polymer. The morphology of the surface of the ceramic is characterized by its visual appearance, its roughness, and/or the size and arrangement of particular morphological features such as local maxima. In embodiments, the surface is characterized by definable sub-micron sized grains. Referring particularly to FIG. 8A, for example, in embodiments, the grains have a length, L, of the of about 50 to 500 nm, e.g. about 100-300 nm, and a width, W, of about 5 nm to 50 nm, e.g. about 10-15 nm. The grains have an aspect ratio (length to width) of about 5:1 or more, e.g. 10:1 to 20:1. The grains overlap in one or more layers. The separation between grains can be about 1-50 nm. In particular embodiments, the grains resemble rice grains.

Referring particularly to FIG. 8B, in embodiments, the surface is characterized by a more continuous surface having a series of globular features separated by striations. The striations have a width of about 10 nm or less, e.g. 1 nm or less, e.g. 1 nm or about 0.1 nm. The striations can be generally randomly oriented and intersecting. The depth of the striations is about 10% or less of the thickness of the coating, e.g. about 0.1 to 5%. In embodiments, the surface resembles an orange peel. In other embodiments, the surface has characteristics between high aspect ratio definable grains and the more continuous globular surface. For example, the surface can include low aspect ratio lobes or thin planar flakes. The morphology type is visible in FESEM images at 50 KX.

The roughness of the surface is characterized by the average roughness, Sa, the root mean square roughness, Sq, and/or the developed interfacial area ratio, Sdr. The Sa and Sq parameters represent an overall measure of the texture of the surface. Sa and Sq are relatively insensitive in differentiating peaks, valleys and the spacing of the various texture features. Surfaces with different visual morphologies can have similar Sa and Sq values. For a surface type, the Sa and Sq parameters indicate significant deviations in the texture characteristics. Sdr is expressed as the percentage of additional surface area contributed by the texture as compared to an ideal plane the size of the measurement region. Sdr further differentiates surfaces of similar amplitudes and average roughness. Typically Sdr will increase with the spatial intricacy of the texture whether or not Sa changes.

In embodiments, the ceramic has a defined grain type morphology. The Sdr is about 30 or more, e.g. about 40 to 60. In addition or in the alternative, the morphology has an Sq of about 15 or more, e.g. about 20 to 30. In embodiments, the Sdr is about 100 or more and the Sq is about 15 or more. In other embodiments, the ceramic has a globular type surface morphology. The Sdr is about 20 or less, e.g. about 8 to 15. The Sq is about 15 or less, e.g. about less than 8 to 14. In still other embodiments, the ceramic has a morphology between the defined grain and the globular surface, and Sdr and Sq values between the ranges above, e.g. an Sdr of about 1 to 200 and/or an Sq of about 1 to 30.

In particular embodiments, the ceramic is iridium oxide. Other suitable ceramics include metal oxides and nitrides, such as of iridium, zirconium, titanium, hafnium, chromium, niobium, tantalum, ruthenium, platinum and aluminum. The ceramic can be crystalline, partly crystalline or amorphous. The ceramic can be formed entirely of inorganic materials or a blend of inorganic and organic material (e.g. a polymer). In other embodiments, the morphologies described herein can be formed of metal. As discussed above, different ceramic materials can be provided in different regions of a stent. For example, different materials may be provided on different surfaces of the depression or ridge. A rougher, defined grain material may be provided on the interior surface to, e.g. enhance adhesion while a material with globular features can be provided on the exterior surfaces to enhance endothelialization. Different materials may also be provided on different stent surfaces. A rougher, defined grain material may be provided on the abluminal surface to, e.g. enhance adhesion while a material with globular features can be provided on the adluminal surface to enhance endothelialization. Further discussion of ceramic morphology including suitable methods for characterizing morphologies and computing roughness parameters is provided in U.S. patent application Ser. No. 11/752,736, [Attorney Docket No. 10527-801001], filed May 23, 2007, and U.S. patent application Ser. No. 11/752,772, [Attorney Docket No. 10527-805001], filed May 23, 2007.

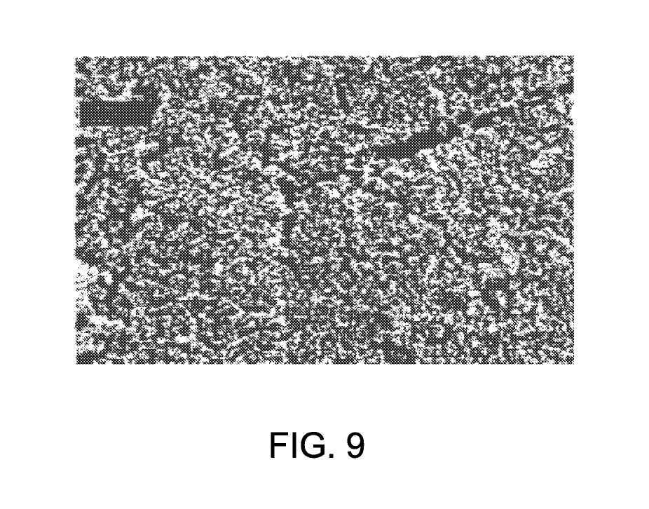

Referring to FIG. 9, a photomicrograph of an etched metal surface is provided. The surface has a rough texture with the same roughness value ranges as described above with respect to ceramic coating embodiments. In embodiments, the roughness can be carried out by electrolytic etching. Platinum enhanced radio-opacity stainless steel (PERSS) in 0.5 molar sulfuric acid using a sawtooth waveform. Voltage was scanned between −240 mV to 1.26 V versus a saturated calomel reference electrode, at 100 mV/sec, the voltage was scanned up and down 30 times. The bath was operated at room temperature without solution agitation. The result is a nanometer scale porous platinum surface. For stainless steel, a pulsed potential square wave etching is used in 5 molar sulfuric acid at 140° F. without agitation, 1 volt for 0.1 seconds, −0.4 volts for 0.01 seconds (voltages measured against the saturated calomel electrode), repeated for 20 minutes. The result is a porous oxide containing Cr and Fe oxides.

In embodiments, the drug is provided directly into the depression or channel without a polymer. In other embodiments, multiple layers of polymer can be provided into the depression or channel. Such multiple layers are of the same or different polymer materials. For example, a biostable polymer such as parylene, Teflon can be first applied on top of the ceramic or metal coating before the drug-containing polymer is applied onto it to, e.g., further enhance adherence of the drug-containing polymer, e.g., a bioerodible polymer to the depression or channel. Examples of bioerodible polymers include polylactic acid (PLA), polylactic glycolic acid (PLGA), polyanhydrides (e.g., poly(ester anhydride)s, fatty acid-based polyanhydrides, amino acid-based polyanhydrides), polyesters, polyester-polyanhydride blends, polycarbonate-polyanhydride blends, and/or combinations thereof. Upon contacting the body fluid during stent delivery or when the stent is placed in desired location, the bioerodible polymer may swell and the volume can increase, e.g., to about twice of its original volume. Unless otherwise defined, the thickness of the polymer means the “dry” thickness in this disclosure. The depression or channel (e.g., one that has lips or ledges) also helps confine the bioerodible polymer in place even if polymer adhesion weakens upon swelling.

The ceramic or metal material can also be selected for compatibility with a particular polymer coating to, e.g. enhance adhesion. For example, for a hydrophilic polymer, the surface chemistry of the ceramic is made more hydrophilic by e.g., increasing the oxygen content, which increases polar oxygen moieties, such as OH groups. Drug eluting polymers may be hydrophilic or hydrophobic. The terms “drug-containing polymer”, “drug eluting polymer” and other related terms may be used interchangeably herein and include, but are not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics such as polystyrene and copolymers thereof with other vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers, polyethylene oxides, glycosaminoglycans, polysaccharides, polyesters including polyethylene terephthalate, and polybutylene suucinate adipate (PBSA), polyacrylamides, polyethers, polyether sulfone, polycarbonate, polyalkylenes including polypropylene, polyethylene and high molecular weight polyethylene, halogenerated polyalkylenes including polytetrafluoroethylene, natural and synthetic rubbers including polyisoprene, polybutadiene, polyisobutylene and copolymers thereof with other vinyl monomers such as styrene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present disclosure. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. U.S. Pat. No. 5,091,205 describes medical devices coated with one or more polyiocyanates such that the devices become instantly lubricious when exposed to body fluids. In embodiments, a suitable polymer is polyacrylic acid, available as HYDROPLUS® (Boston Scientific Corporation, Natick, Mass.), and described in U.S. Pat. No. 5,091,205, the disclosure of which is hereby incorporated herein by reference. Another polymer can be a copolymer of polylactic acid and polycaprolactone. Suitable polymers are discussed in U.S. Publication No. 2006/0038027.

The terms “therapeutic agent”, “pharmaceutically active agent”, “pharmaceutically active material”, “pharmaceutically active ingredient”, “biologically active substance”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), immunosuppressants (e.g., everolimus, tacrolimus), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application Nos. 2005/0019265 and 2005/0251249. A functional molecule, e.g. an organic, drug, polymer, protein, DNA, and similar material can be incorporated into groves, pits, void spaces, and other features of the ceramic.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6A1-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, and urethral lumens.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprostheses or medical devices, such as catheters, guide wires, and filters.

All publications, patent applications, and patents, are incorporated by reference herein in their entirety.

Still other embodiments are in the following claims.

What is claimed is:

1. An endoprosthesis, comprising:
a channel on a surface region,
the channel including a ceramic coating on at least a portion of its interior surface,
the coating having a defined grain morphology.

2. The endoprosthesis of claim 1, wherein the channel includes a polymer containing a drug adhered to the ceramic.

3. The endoprosthesis of claim 2, wherein the polymer is swellable on exposure to body fluid.

4. The endoprosthesis of claim 1, wherein the coating has an Sdr of about 3 or more.

5. The endoprosthesis of claim 1, wherein the ceramic includes oxides and nitrides of iridium, titanium, zirconium, hafnium, niobium, tantalum, ruthenium, platinum, and aluminum.

6. The endoprosthesis of claim 1, wherein the ceramic is IROX.

7. The endoprosthesis of claim 1, wherein the coating has a thickness of about 10 to 500 m.

8. The endoprosthesis of claim 1, wherein the surface region is the abluminal surface of a stent wall.

9. The endoprosthesis of claim 8, wherein the channel has a depth of about 50% or less of the thickness of the stent wall.

10. The endoprosthesis of claim 9, wherein the polymer has a thickness smaller than the depth of the channel.

11. A method of forming an endoprosthesis, comprising:
forming a channel on the endoprosthesis,
treating the interior surface of the channel such that at least a portion of the
surface has an Sdr of 30 or greater, and
applying a polymer containing a drug to the channel.

12. The method of claim 11, comprising forming the channel by a laser ablation process.

13. The method of claim 11, comprising forming the channel in the body of the endoprosthesis.

14. The method of claim 11, comprising forming a coating on the endoprosthesis and forming the channel in the coating.

15. The method of claim 14, wherein the coating is a ceramic.

16. The method of claim 15, wherein the ceramic is formed by PLD.

17. The method of claim 11, comprising applying the polymer by dipping, spraying, or vapor deposition.

18. The method of claim 11, comprising treating the interior surface by etching.

19. The method of claim 11, comprising treating the interior surface by depositing a ceramic layer.

20. The method of claim 19, comprising applying the ceramic layer by PLD.

21. The method of claim 19, wherein the ceramic has a defined grain morphology.

22. The method of claim 21, wherein the ceramic is IROX.
